(19) United States
(12) Patent Application Publication
(10) Pub. No.: US 2022/0133529 A1
(43) Pub. Date: May 5, 2022

(54) FLEXIBLE COLD PACKS

(71) Applicant: Vive Health LLC, Naples, FL (US)

(72) Inventor: Daniel Fleming, Naples, FL (US)

(21) Appl. No.: 17/083,643

(22) Filed: Oct. 29, 2020

(51) Int. Cl. A61F 7/10

(52) U.S. Cl. CPC A61F 7/10; A61F 2007/0253; A61F 2007/108

(57) ABSTRACT

A flexible cold pack includes a cell layer and a flexible layer. The cell layer includes a plurality of cells that are coupled to one another. Each cell of the plurality of cells includes a first liquid associated with a first freezing temperature. The flexible layer is disposed adjacent to the cell layer such that the flexible layer spans the plurality of cells of the cell layer. The flexible layer includes a second liquid or flexible semi-solid disposed therein. The second liquid or flexible semi-solid is associated with a second freezing temperature that is lower than the first freezing temperature.

### BACKGROUND

Many individuals employ cryotherapy as a means of achieving pain relief and/or managing swelling. Cryotherapy often involves reducing the temperature of a part of a body by externally applying a cooling stimulus to a part of the body that absorbs heat from the part of the body. By way of example, cryotherapy can be applied via ice packs, coolant sprays, ice massage, ice baths, etc. Cryotherapy is often utilized in a self-directed manner and can also be prescribed and/or utilized by medical practitioners to treat and/or assist in recovery from certain conditions. For example, users may utilize cryotherapy to achieve pain relief and/or manage swelling associated with runner's knee, tendonitis, sprains, arthritis, postoperative pain (e.g., from hip or knee replacement), lower back pain, and/or others.

Cryotherapy is used to treat many conditions associated with parts of the body that form curved, rounded, and/or non-flat surfaces (e.g., knees, ankles, elbows, shoulders, hips, and/or other body parts). Some cryotherapy products have been developed that can form over curved, rounded, and/or non-flat bodily surfaces. However, such cryotherapy devices typically fail to provide a cooling effect for a significant time period (e.g., longer than thirty minutes). In contrast, some cryotherapy products that are configured to provide a cooling effect for a significant time period (e.g., longer than thirty minutes) are unable to form over curved, rounded, and/or non-flat bodily surfaces.

For at least the foregoing reasons, there is an ongoing need and desire for flexible cold packs that can provide a cooling effect for a significant time period.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Implementations of the present disclosure extend at least to flexible cold packs.

Some embodiments provide a flexible cold pack that includes a cell layer and a flexible layer. The cell layer includes a plurality of cells that are coupled to one another. Each cell of the plurality of cells includes a first liquid associated with a first freezing temperature. The flexible layer is disposed adjacent to the cell layer such that the flexible layer spans the plurality of cells of the cell layer. The flexible layer includes a second liquid or flexible semi-solid disposed therein. The second liquid or flexible semi-solid is associated with a second freezing temperature that is lower than the first freezing temperature.

Some embodiments provide a flexible cold pack that includes an envelope formed from flexible material and a plurality of cells disposed within the envelope. Each cell of the plurality of cells disposed within the envelope includes a first liquid associated with a first freezing temperature. The flexible cold pack also includes a second liquid or flexible semi-solid disposed within the envelope among the plurality of cells. The second liquid or flexible semi-solid is associated with a second freezing temperature that is lower than the first freezing temperature.

Some embodiments provide a flexible cold pack that includes an envelope formed from a flexible material, a liquid or flexible semi-solid disposed within the envelope, and an insulating layer disposed within the envelope on at least one interior side of the envelope.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings.

FIG. 1 illustrates a front perspective view of a flexible cold pack that includes a cell layer and two flexible layers;

FIG. 2 illustrates a top plan view of the flexible cold pack of FIG. 1;

FIG. 3 illustrates a left side view of the flexible cold pack of FIGS. 1 and 2;

FIG. 4 illustrates a left side view of a flexible cold pack that includes a cell layer, a flexible layer, and an insulating layer;

FIG. 5 illustrates a left side view of a flexible cold pack that includes a flexible layer and a cell layer with cells that include multiple compartments;

FIG. 6 illustrates a front perspective view of a flexible cold pack that includes cells disposed within a liquid or semi-solid medium;

FIG. 7 illustrates a left side view of the flexible cold pack of FIG. 6;

FIG. 8 illustrates a front perspective view of a flexible cold pack that includes a flexible envelope that houses a liquid or semi-solid medium and an insulating layer; and

FIG. 9 illustrates a left side view of the flexible cold pack of FIG. 8.

### DETAILED DESCRIPTION

Implementations of the present disclosure extend at least to flexible cold packs.

In some implementations, a flexible cold pack that includes a cell layer and a flexible layer. The cell layer includes a plurality of cells that are coupled to one another. Each cell of the plurality of cells includes a first liquid associated with a first freezing temperature. The flexible layer is disposed adjacent to the cell layer such that the flexible layer spans the plurality of cells of the cell layer. The flexible layer includes a second liquid or flexible semi-solid disposed therein. The second liquid or flexible semi-solid is associated with a second freezing temperature that is lower than the first freezing temperature.

Some embodiments provide a flexible cold pack that includes an envelope formed from flexible material and a plurality of cells disposed within the envelope. Each cell of the plurality of cells disposed within the envelope includes a first liquid associated with a first freezing temperature. The flexible cold pack also includes a second liquid or flexible semi-solid disposed within the envelope among the plurality of cells. The second liquid or flexible semi-solid is associated with a second freezing temperature that is lower than the first freezing temperature.

At least some implementations of the present disclosure comprise a flexible cold pack that includes an envelope formed from a flexible material, a liquid or flexible semi-solid disposed within the envelope, and an insulating layer disposed within the envelope on at least one interior side of the envelope.

Those skilled in the art will recognize, in view of the present disclosure, that at least some of the disclosed embodiments may address various shortcomings associated with conventional cold packs used for cryotherapy.

For instance, at least some flexible cold packs of the present disclosure include different media that work in combination to facilitate heat absorption from a part of a body. By way of example, at least some cold packs of the present disclosure include cells filled with one type of medium (e.g., water), and the cells are disposed within another medium (e.g., silica gel). In preparation for using a flexible cold pack of the present disclosure, a user may place the flexible cold pack in an available cooling environment (e.g., a household or commercial freezer). After the flexible cold pack substantially reaches the temperature of the cooling environment, the medium within the cells may freeze into a solid structure, while the medium surrounding the cells may remain in a flexible state (e.g., a liquid or semi-solid state).

Subsequently, while using the flexible cold pack for cryotherapy, the frozen medium within the cells may undergo a phase change from solid to liquid. Facilitating such a phase change during cryotherapy may allow the flexible cooling pack to provide a cooling effect for a substantial time period (e.g., as compared with cooling products that omit any components that solidify in preparation for cryotherapy).

Furthermore, the benefits of facilitating the phase change as described hereinabove may be realized while preserving flexible characteristics for the cold packs of the present disclosure. In some instances, the cells that house the medium that solidifies in preparation for cryotherapy are uncoupled from one another, allowing the cells, even when solidified, to freely move within the surrounding flexible medium of the cooling pack. In some instances, the cells that house the medium that solidifies in preparation for cryotherapy are coupled to one another in a manner that allows the cells, even when solidified, to move relative to one another during cryotherapy (e.g., the cells may be arranged in a cell layer with each cell spatially offset from its neighbors).

In addition, some flexible cold packs of the present disclosure include insulating layers and/or components that allow the flexible cold packs to focus heat absorption on particular portions of the flexible cold packs. For example, some flexible cold packs of the present disclosure may include an insulating layer on only one side of the cold pack, allowing the cold pack to primarily absorb heat from the side of the cold pack that omits the insulating layer. In this regard, at least some flexible cold packs of the present disclosure may be operable to focus cooling effects on particular portions of a user's body, while minimizing heat absorption from the surrounding environment (which may prolong the cooling effect provided by the flexible cold pack).

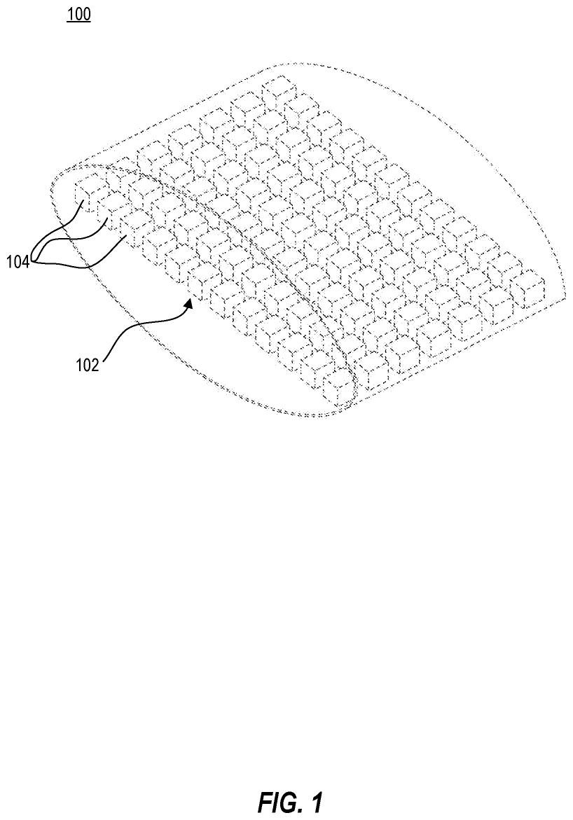

Having described some of the various high-level features and benefits of the disclosed embodiments, attention will now be directed to FIGS. 1 through 9. These Figures illustrate various supporting illustrations related to the disclosed embodiments.

FIG. 1 illustrates a front perspective view of a flexible cold pack 100, according to the present disclosure. FIG. 1 illustrates an implementation in which the flexible cold pack 100 includes a cell layer 102. The cell layer 102, as represented in FIG. 1, comprises a plurality of cells 104. Each cell 104 may form an individual fluid-tight compartment that is configured to retain a medium, such as a liquid medium or a semi-solid medium (e.g., a gel medium).

In some examples, the medium disposed within each cell 104 of the cell layer 102 is water. In other examples, the medium disposed within each cell 104 of the cell layer is any safe, freezable liquid. Thus, a user may place the flexible cold pack 100 within a cooling environment, such as a freezer, in preparation for using the flexible cold pack 100 for cryotherapy. After some time, the temperature of the flexible cold pack 100 and the temperature of the water disposed within the cells 104 may reach the freezing temperature of the water, and the water within the cells 104 may solidify. As noted hereinabove, the frozen state of the medium within the cells 104 may prolong the cooling effects provided by the flexible cold pack 100 when properly used for cryotherapy.

The cells 104 may be formed of a flexible material suitable for the cells 104 to accommodate the medium disposed therein regardless of the state of the medium. For example, where the medium comprises water, the cells 104 may be configured to accommodate the water as a liquid and may be configured to flex and expand with the water as the water expands and freezes into solid ice. Furthermore, during heat absorption (e.g., during cryotherapy), the cells may also be configured to retract as the water shrinks from its frozen state into a liquid state.

Although FIG. 1 illustrates the cells 104 of the cell layer 102 of the flexible cold pack as comprising a substantially cube-like or rectangular prism-like shape, it should be noted that a cell 104 may comprise any suitable shape in accordance with the present disclosure. By way of non-limiting example, one or more of the cells 104 may comprise a spherical, ellipsoidal, cylindrical, conical, toroidal, pyramidal, polyhedral, polygonal prism, and/or any other regular or irregular shape. Furthermore, it will be appreciated, in view of the present disclosure, that the shapes of the cells 104 need not be uniform in shape, size, or orientation across the entire cell layer 102.

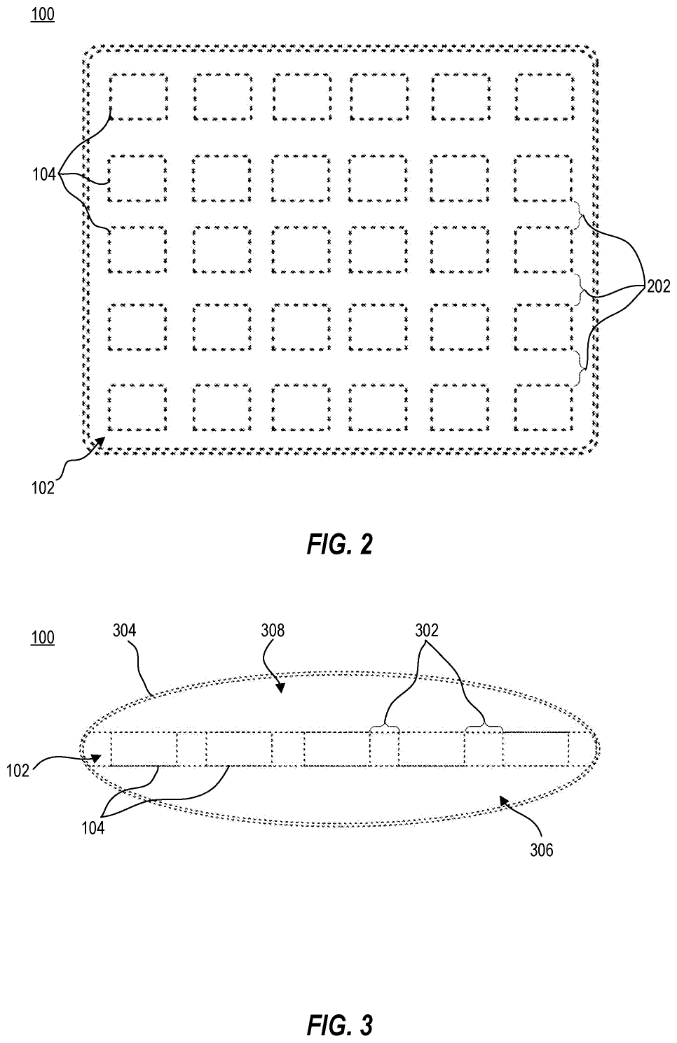

FIG. 2 illustrates a top plan view of the flexible cold pack 100 of FIG. 1. FIG. 2 demonstrates that, in some implementations, at least some of the cells 104 of the cell layer 102 of the flexible cold pack 100 are offset from one another in a first dimension, forming space 202 between the various cells 104 of the cell layer 102. FIG. 3 illustrates a left side view of the flexible cold pack of FIGS. 1 and 2. Similarly, FIG. 3 demonstrates that, in some implementations, at least some of the cells 104 of the cell layer 102 of the flexible cold pack 100 are offset from one another in a second dimension (the second dimension being perpendicular to the first dimension), forming space 302 between the various cells 104 of the cell layer 102.

In some instances, the offsets between the various cells 104 of the cell layer 102 enable the cells 104 of the cell layer 102 to form an at least partially flexible planar structure or arrangement, which may enable the cell layer 102 to bend and/or shape to the contours of a non-flat portion of a user's body as the flexible cold pack 100 is applied during cryotherapy, even when the cells 104 house a solid medium (e.g., frozen water). FIG. 3 also illustrates that at least some of the cells 104 of the cell layer 102 are coupled together, which may allow the cells 104 to retain a layer-like structure even while being curved to fit a non-flat portion of a user's body during cryotherapy. Retaining the layer-like structure may allow the cell layer 102 to at least partially retain its position relative to other portions of the flexible cold pack 100 to control heat absorption of the cells 104 (e.g., by keeping the cell layer 102 centrally located within the flexible cold pack 100).

One will note, in view of the present disclosure, that the offsets that form spaces 202 and 302 illustrated in FIGS. 2 and 3, respectively, are illustrative only and non-limiting. For example, the offsets in any of the dimensions described may be uniform or non-uniform as between different cells 104 of the cell layer, in accordance with implementations of the present disclosure. Furthermore, although FIGS. 1 through 3 focus, in at least some respects, on implementations in which a flexible cold pack 100 includes a single cell layer 102, a flexible cold pack 100 may comprise multiple cell layers, and an offset may exist in a third dimension between the cells of different cell layers (e.g., the third dimension being perpendicular to both the first dimension described hereinabove with reference to FIG. 2 and the second dimension described hereinabove with reference to FIG. 3).

FIG. 3 further illustrates that, in some implementations, the flexible cold pack 100 comprises an envelope 304. The envelope 304 may comprise any suitable flexible material that allows the flexible cold pack 100 to form to the contours of curved, rounded, and/or non-flat portions of user bodies. For example, in some instances, the envelope 304 may comprise flexible vinyl.

FIG. 3 also demonstrates that the flexible cold pack 100 may comprise one or more flexible layers, and the one or more flexible layers may be disposed within the envelope 304 adjacent to the cell layer 102 (e.g., being disposed on opposing sides of the cell layer 102). For instance, FIG. 3 shows that the flexible cold pack 100 includes a first flexible layer 306 disposed within the envelope 304 on a first side of the cell layer 102, as well as a second flexible layer 308 disposed within the envelope 304 on a second side of the cell layer 102 (where the second side of the cell layer 102 is opposite to the first side of the cell layer 102).

As is evident in FIG. 3, the first flexible layer 306 and the second flexible layer 308 are arranged to span or extend in abutment along at least some of the cells 104 of the cell layer 102. The first flexible layer 306 and/or the second flexible layer 308 may be configured to house a medium (e.g., a liquid or flexible semi-solid) that is different than the medium housed within the cells 104 of the cell layer 102. For example, the first flexible layer 306 and/or the second flexible layer 308 may comprise, as a medium, silica gel, sodium polyacrylate, hydroxyethyl cellulose, combinations thereof, and/or other liquid or flexible semi-solid matter (e.g., gel).

The medium disposed within the first flexible layer 306 and/or the second flexible layer 308 may be associated with a freezing temperature that is different than the freezing temperature associated with the medium disposed within the cells 104 of the cell layer 102. For example, where the cells 104 house a water medium and the first flexible layer 306 and/or second flexible layer 308 house a silica gel medium, the silica gel may be associated with a freezing temperature that is lower than the freezing temperature of water.

In this way, when the temperature of the flexible cold pack 100 is brought to the freezing point of water (e.g., within a conventional freezer in preparation for cryotherapy), the water disposed within the cells 104 of the cell layer 102 may freeze while the silica gel within the first flexible layer 306 and/or the second flexible layer 308 may remain in a flexible semi-solid state and be able to form over curved, rounded, and/or non-flat portions of a user's body.

Thus, during cryotherapy, the first flexible layer 306 and/or the second flexible layer 308 (which may be regarded as outer layers) may form to non-flat user body parts to allow the medium/media disposed therein to provide a cooling effect to the body part, while the frozen medium disposed within the cells 104 of the cell layer 102 may undergo a phase change during cryotherapy to prolong the cooling effect provided by the medium/media of the flexible layers that surround the cell layer 102. Furthermore, as noted above, the cell layer 102 may be configured to at least partially bend and/or reshape with the first flexible layer 306 and/or the second flexible layer 308 to provide the prolonged cooling effect mentioned above without significantly restricting the flexibility of the flexible cold pack 100.

In some implementations, at least some of the spaces formed between the cells 104 of the cell layer 102 (e.g., spaces 202 and/or 302 defined by the offsets described hereinabove) provide or form one or more conduits configured to allow media to flow from the first flexible layer 306 to the second flexible layer 308 and vice versa. In some implementations, at least some of the spaces formed between the cells 104 of the cell layer 102 may prevent media from passing between the first flexible layer 306 and the second flexible layer 308. For example, the various cells 104 of the cell layer 102 may be connected or coupled to one another via non-porous segments of material that form a single layer between the first flexible layer 306 and the second flexible layer 308.

In other instances, the non-porous segments of material may form a dual-layer interface between the first flexible layer 306 and the second flexible layer 308 (as depicted in FIG. 3), forming, in effect, additional cells from the space that intervenes between the cells 104. In such implementations, the cell layer 102 may be regarded as having two types of cells: one type corresponding to the cells 104 described hereinabove, and another type that includes a flexible medium (e.g., a liquid, gas, or semi-solid) with a lower freezing point than the medium of the cells 104 (e.g., water, in some instances) to allow the cell layer 102 to retain flexible qualities when the medium within the cells 104 is solidified or frozen.

It should be noted that the first flexible layer 306 and the second flexible layer 308 of the flexible cold pack 100 may comprise the same liquid or flexible semi-solid medium disposed therein or may comprise different liquid or flexible semi-solid media disposed therein.

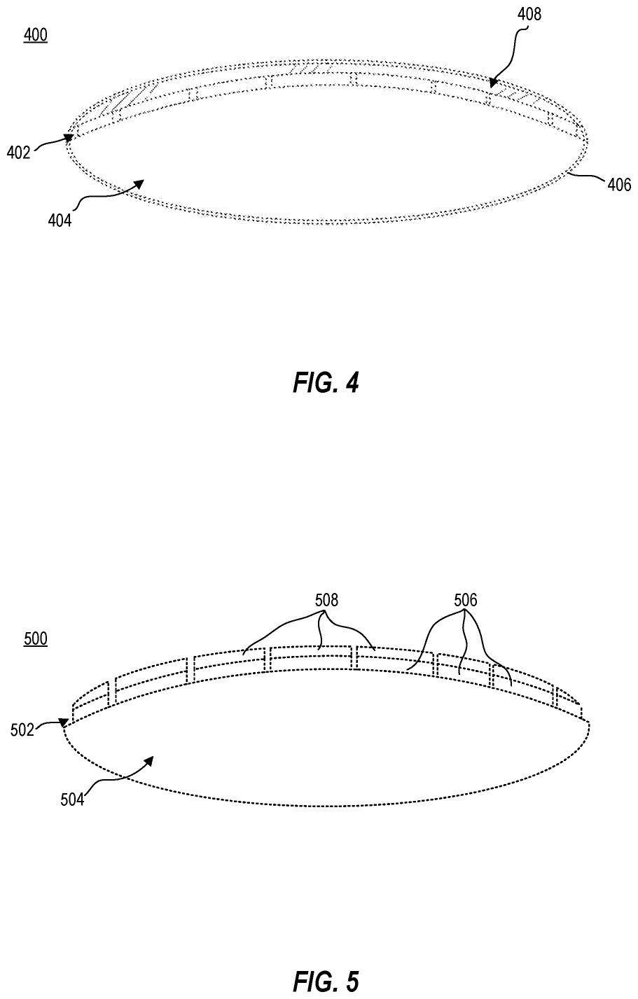

Although FIGS. 1 through 3 focus, in at least some respects, on a flexible cold pack 100 that includes a cell layer 102 and two flexible layers (e.g., first flexible layer 306 and second flexible layer 308), other configurations are within the scope of this disclosure. For example, FIG. 4 illustrates a left side view of another embodiment of a flexible cold pack 400. That includes a cell layer 402 and only a single flexible layer 404 arranged within an envelope 406 adjacent to the cell layer 402. FIG. 4 illustrates that the cells of the cell layer 402 comprise a different geometry than the cells 104 of the cell layer 102 described hereinabove for the flexible cold pack 100 of FIGS. 1 through 3.

In some implementations, space formed between the cells of the cell layer 402 may be configured in fluid communication with the flexible layer 404, notwithstanding the lack of a second flexible layer on the opposing side of the cell layer 402. Such an arrangement may allow the cells of the cell layer 402 to absorb heat from the medium disposed within the flexible layer 404 through multiple sides of the cells.

FIG. 4 also demonstrates that, in some implementations, a flexible cold pack 400 may comprise an insulating layer 408 disposed adjacent to the cell layer 402. In some instances, the insulating layer 408 is disposed on an opposing side of the cell layer 402 relative to the flexible layer 404, such that the insulating layer 408 and the flexible layer 404 are arranged on opposite sides of the cell layer 402.

In some instances, the insulating layer 408 includes an insulation envelope that encompasses insulating matter. Similar to the flexible layer 404, the insulating envelope of the insulating layer 408 may span multiple cells of the cell layer 402 of the flexible cold pack 400. In this way, the insulating layer 408 may allow the flexible cold pack 400 to primarily absorb heat from the side of the flexible cold pack 400 that omits the insulating layer 408 (i.e., the side of the flexible cold pack 400 where the outermost layer comprises the flexible layer 404). Such a configuration may allow the flexible cold pack 400 to focus cooling effects on particular portions of a user's body, while minimizing heat absorption from the surrounding environment (which may prolong the cooling effect provided by the flexible cold pack 400).

The insulation envelope of the insulating layer 408 may comprise any insulating material known in the art, such as, by way of non-limiting example, air or another gas, cellulose, mineral wool, polyurethane, polystyrene, fiberglass, combinations thereof, and/or other materials.

Although FIG. 4 illustrates the insulating layer 408 as comprising a single insulation envelope and as disposed within the envelope 406 that houses the cell layer 402 and the flexible layer 404, other configurations are within the scope of this disclosure. For example, FIG. 5 illustrates a left side view of another embodiment of a flexible cold pack 500. Similar to the flexible cold packs 100 and 400 described hereinabove with reference to FIGS. 1 through 4, the flexible cold pack 500 includes a cell layer 502 and at least one flexible layer 504. However, as is evident from FIG. 5, the cells of the cell layer 502 include multiple compartments.

For example, the cells of the cell layer 502 each comprise a first compartment 506 and a second compartment 508. In some instances, the first compartment 506 of each cell comprises the medium that is selected to freeze or solidify in preparation for cryotherapy (e.g., water), and the second compartment of each cell comprises insulating matter. FIG. 5 illustrates an implementation in which the first compartment 506 of each cell is disposed in abutment with the flexible layer 504, and each second compartment 508 of each cell is disposed in abutment with a respective first compartment 506 (e.g., on an opposing side of the respective first compartment 506 relative to the flexible layer 504).

In some instances, the configuration for a flexible cold pack 500 shown in FIG. 5 may enable the flexible cold pack to provide the benefits described hereinabove of providing a long cooling effect (e.g., facilitated at least in part by including a medium within the first compartments 506 of the cells of the cell layer 502 that freezes in preparation for cryotherapy) and minimizing heat absorption from a surrounding environment (e.g., facilitated at least in part by including insulating matter within the second compartments 508 of the cells of the cell layer 502) while achieving a high degree of flexibility to form to non-flat bodily surfaces. For instance, because the flexible cold pack 500 omits a unitary insulating layer (e.g., in contrast with flexible cold pack 400 of FIG. 4) and instead provides multi-compartment cells that each include insulating material, the cells of the cell layer 502 may be configured to achieve greater curvature relative to one another without being constrained by a common outer insulating layer.

By way of example, when fitting the flexible layer 504 of the flexible cold pack 500 over a highly rounded bodily surface such as an elbow, the cells of the cell layer 502 may be able to bend about the elbow with the flexible layer 504, in particular because the cells are not additionally held together on an outer side of the flexible cold pack 500 by a shared insulating layer (e.g., insulating layer 408 from flexible cold pack 400 of FIG. 4).

Although FIG. 5 only depicts the second compartments 508 of the cells of the cell layer 502 as abutting only one side of the respective first compartments 506 of the cells of the cell layer 502, the second compartments 508 may, in some implementations, abut multiple sides of the respective first compartments 506 to provide an insulating effect thereover. In other configurations, the cells of the cell layer 502 may comprise multiple compartments that house insulating matter, and the multiple compartments may abut multiple sides of corresponding first compartments 506 of the cells.

As noted above FIG. 5 illustrates an implementation of a flexible cold pack 500 that omits a common envelope that houses all of the layers of the flexible cold pack. For instance, the flexible layer 504 of the flexible cold pack 500 may comprise a flexible envelope that houses the medium configured to remain flexible when the flexible cold pack 500 is cooled in preparation for cryotherapy, whereas the cells of the cell layer 502 may comprise one or more separate cell envelopes. The various envelopes may comprise flexible material, such as flexible vinyl.

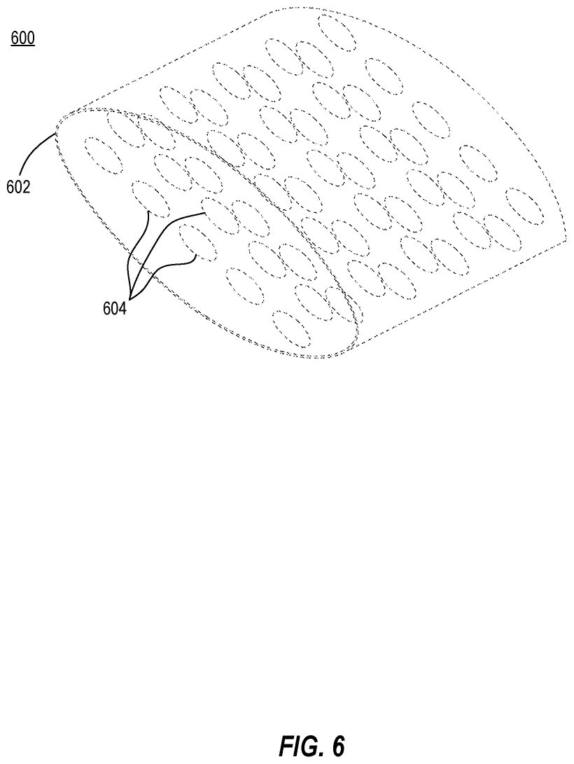

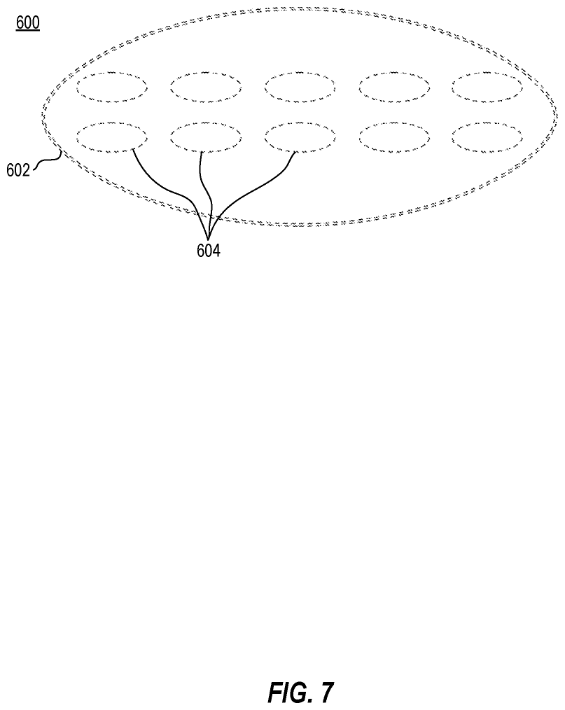

Although the foregoing description has focused, in at least some respects, on implementations in which the cells of a flexible cold pack are at least partially coupled together to form a layer-like or matrix-like structure, other configurations are within the scope of this disclosure. For instance, FIG. 6 illustrates a front perspective view of another embodiment of a flexible cold pack 600, and FIG. 7 illustrates a left side view of the flexible cold pack 600 of FIG. 6.

The flexible cold pack 600 includes an envelope 602 formed from a flexible material (e.g., flexible vinyl). The envelope 602 houses a medium configured to remain in a flexible state (e.g., a liquid or flexible semi-solid state) when the flexible cold pack 600 is cooled in preparation for administering cryotherapy (e.g., cooled within a freezer). As shown in FIGS. 6 and 7, the flexible cold pack 600 also includes a plurality of cells 604 disposed within the envelope 602 among the medium housed within the envelope 602. The cells 604 of the flexible cold pack 600, as illustrated in FIGS. 6 and 7, are uncoupled from one another (e.g., in contrast with the cells of the layers of cells described hereinabove with reference to FIGS. 1 through 5).

Each of the plurality of cells 604 of the flexible cold pack 600 may comprise a liquid medium disposed therein that is configured to freeze or solidify when the flexible cold pack 600 is cooled in preparation for administering cryotherapy. Thus, during cryotherapy, the flexible cold pack 600 may provide a prolonged cooling effect via the frozen medium within the cells 604 while retaining flexible characteristics via the flexible medium surrounding the cells 604 within the envelope 602. Furthermore, the uncoupled nature of the cells 604 allows the cells 604 to freely move within the envelope 602 among the medium surrounding the cells 604 and may, in some instances, allow the flexible cold pack 600 to achieve greater flexibility than at least some other embodiments described herein.

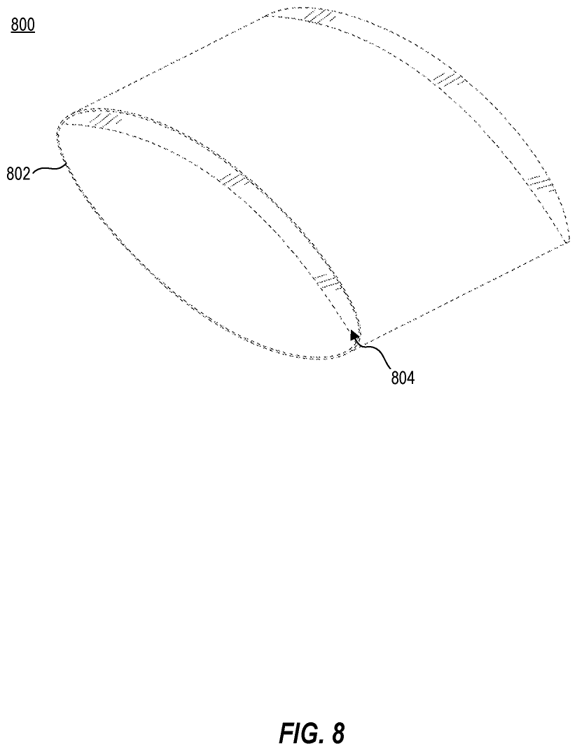

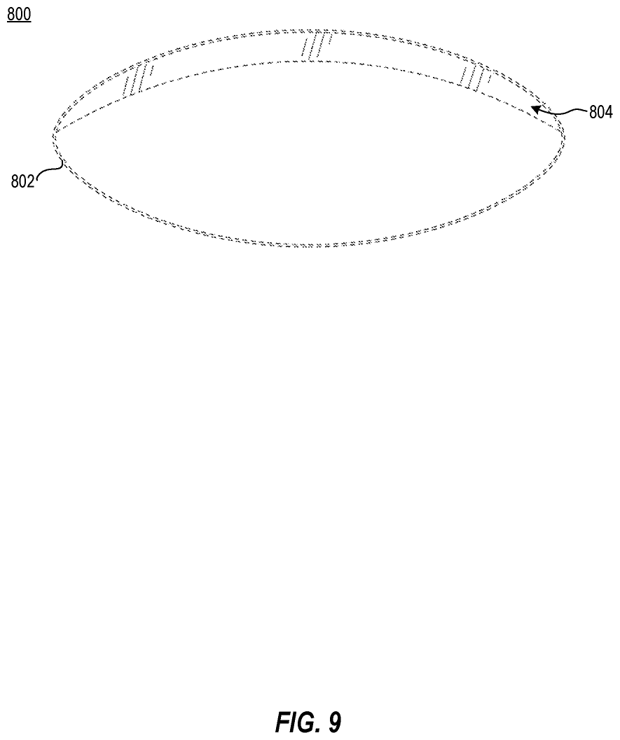

FIG. 8 illustrates a front perspective view of yet another embodiment of a flexible cold pack 800, and FIG. 9 illustrates a left side view of the flexible cold pack 800 of FIG. 8. The flexible cold pack 800 includes an envelope 802 formed from a flexible material (e.g., flexible vinyl). The envelope 802 houses a medium configured to remain in a flexible state (e.g., a liquid or flexible semi-solid state) when the flexible cold pack 800 is cooled in preparation for administering cryotherapy (e.g., cooled within a freezer).

The flexible cold pack also includes an insulating layer 804 arranged within the envelope 802 and abutting at least one interior side of the envelope 802. Such a configuration may allow the flexible cold pack 800 to focus cooling effects on particular portions of a user's body, while minimizing heat absorption from the surrounding environment (which may prolong the cooling effect provided by the flexible cold pack 800). The insulating layer may comprise any insulating matter known in the art.

It will be appreciated, in view of the present disclosure, that various components of flexible cold packs may be combined with one another, even where such components were not described with reference to the same Figure or particular embodiment. By way of non-limiting example, although the flexible cold pack 100 of FIGS. 1, 2, and 3 is illustrated without an insulating layer, a flexible cold pack 100 may include an insulating layer in accordance with implementations of the present disclosure.

As another example, although the flexible cold pack 100 of FIGS. 1, 2, and 3, the flexible cold pack 400 of FIG. 4, and the flexible cold pack 500 of FIG. 5 are illustrated without uncoupled cells that are able to freely move throughout an envelope within another medium, such uncoupled cells may be included in a flexible cold pack 100, flexible cold pack 400, and/or flexible cold pack 500 in accordance with implementations of the present disclosure.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A flexible cold pack, comprising:
a cell layer comprising a plurality of cells being coupled to one another, each cell of the plurality of cells comprising a first liquid associated with a first freezing temperature; and
a flexible layer disposed adjacent to the cell layer such that the flexible layer spans the plurality of cells of the cell layer, the flexible layer comprising a second liquid or flexible semi-solid disposed therein, the second liquid or flexible semi-solid being associated with a second freezing temperature that is lower than the first freezing temperature.

2. The flexible cold pack of claim 1, wherein when the flexible cold pack is brought to the first freezing temperature, the first liquid within the plurality of cells of the cell layer freezes into a solid state and the second liquid or flexible semi-solid within the flexible layer remains in a liquid or flexible semi-solid state.

3. The flexible cold pack of claim 2, wherein the cells of the plurality of cells of the cell layer are offset from one another such that the plurality of cells of the cell layer forms an at least partially flexible planar arrangement.

4. The flexible cold pack of claim 3, further comprising an envelope formed from flexible material, wherein the cell layer and the flexible layer are both disposed within the envelope.

5. The flexible cold pack of claim 4, wherein space between the cells of the plurality of cells of the cell layer is configured in fluid communication with the flexible layer.

6. The flexible cold pack of claim 4, further comprising a second flexible layer disposed within the envelope adjacent to an opposing side of the cell layer relative to the flexible layer.

7. The flexible cold pack of claim 6, wherein the second flexible layer comprises the second liquid or flexible semi-solid disposed therein.

8. The flexible cold pack of claim 7, wherein the cell layer comprises one or more conduits configured to allow the second liquid to flow from the flexible layer to the second flexible layer and vice versa.

9. The flexible cold pack of claim 3, further comprising an insulating layer disposed adjacent to an opposing side of the cell layer relative to the flexible layer.

10. The flexible cold pack of claim 9, wherein the insulating layer comprises an insulation envelope comprising insulating matter disposed therein.

11. The flexible cold pack of claim 3, wherein each cell of the plurality of cells comprises a first compartment and a second compartment, each first compartment being disposed in abutment with the flexible layer and each second compartment being disposed in abutment with a respective first compartment on an opposing side of the respective first compartment relative to the flexible layer.

12. The flexible cold pack of claim 11, wherein the first liquid is disposed in each first compartment of each cell of the plurality of cells, and wherein insulating matter is disposed within each second compartment of each of the plurality of cells.

13. The flexible cold pack of claim 11, wherein the flexible layer comprises a flexible envelope, and wherein the second liquid or flexible semi-solid is disposed within the flexible envelope.

14. A flexible cold pack, comprising:
an envelope formed from flexible material;
a plurality of cells disposed within the envelope, each cell of the plurality of cells comprising a first liquid associated with a first freezing temperature; and
a second liquid or flexible semi-solid disposed within the envelope among the plurality of cells, the second liquid or flexible semi-solid being associated with a second freezing temperature that is lower than the first freezing temperature.

15. The flexible cold pack of claim 14, wherein when the flexible cold pack is brought to first freezing temperature, the first liquid within the plurality of cells freezes into a solid state and the second liquid or flexible semi-solid remains in a liquid or flexible semi-solid state.

16. The flexible cold pack of claim 15, wherein the first liquid comprises water.

17. The flexible cold pack of claim 15, wherein the second liquid or flexible semi-solid comprises silica gel, sodium polyacrylate, or hydroxyethyl cellulose.

18. The flexible cold pack of claim 15, wherein each cell of the plurality of cells is uncoupled from one another.

19. The flexible cold pack of claim 15, further comprising an insulating layer disposed within the envelope on at least one interior side of the envelope.

20. A flexible cold pack, comprising:
an envelope formed from flexible material;
a liquid or flexible semi-solid disposed within the envelope; and
an insulating layer disposed within the envelope on at least one interior side of the envelope.
